(19) United States
(12) Patent Application Publication
(10) Pub. No.: US 2026/0137808 A1
(43) Pub. Date: May 21, 2026

(54) ENGINEERED EXOSOMES AND METHODS FOR PRODUCING THE SAME

(71) Applicant: University of Kentucky Research Foundation, Lexington, KY (US)

(72) Inventors: Sheng Tong, Lexington, KY (US); Xiaoyue Yang, Lexington, KY (US)

(21) Appl. No.: 19/395,653

(22) Filed: Nov. 20, 2025

Related U.S. Application Data
(60) Provisional application No. 63/722,829, filed on Nov. 20, 2024.

(51) Int. Cl. A61K 48/00; C12N 9/22; C12N 15/11

(52) U.S. Cl. CPC A61K 48/0041; A61K 48/0058; C12N 9/226; C12N 15/11; C12N 2310/20

(57) ABSTRACT

Methods for producing an engineered exosome (eEXO) that make use of one or more magnetic nanoparticles (MNPs) complexed with a therapeutic payload are provided. The MNP-therapeutic payload complex is loaded into an endosome of a producer cell. The enriched endosome containing the MNP-therapeutic payload complex is extracted from the producer cell and extruded to produce the eEXO. Loading of the MNP-therapeutic payload complex and extraction of the enriched endosome can be facilitated by the application of a magnetic force to the MNP-therapeutic payload complex. A clustered regularly interspersed short palindromic repeat (CRISPR) ribonucleoprotein (RNP) can be used as the therapeutic payload to produce eEXOs useful in gene editing applications. eEXOs are also provided.

### CROSS-REFERENCE TO RELATED APPLICATION

The present application claims priority to U.S. patent application Ser. No. 63/722,829 filed on Nov. 20, 2024, the entire disclosure of which is incorporated herein by reference.

### GOVERNMENT INTEREST

This invention was made with government support under the NIH Common Fund awarded by the National Institutes of Health. The government has certain rights in the invention.

### TECHNICAL FIELD

The presently disclosed subject matter generally relates to artificially engineered exosomes (AREs), which can also be characterized as engineered exosomes (eEXOs). In particular, certain embodiments of the presently disclosed subject matter include eEXOs and methods of eEXO production which may prove useful in gene editing applications.

### BACKGROUND

CRISPR-based genome editing holds tremendous promise for treating human disease by pairing programmable guide ribonucleic acids (gRNAs) with CRISPR-associated protein (Cas) nucleases, CRISPR enables precise deletion, insertion, modification, or regulation of genomic loci, allowing permanent correction of disease-causing mutations. The first CRISPR therapy was approved for sickle cell disease, validating its clinical potential. However, this therapy has traditionally required ex vivo manipulation of hematopoietic stem/progenitor cells (HSPCs), myeloablative conditioning, and reinfusion. While effective, these procedures are costly, invasive, and limited in scope. In vivo gene editing could greatly expand the reach of CRISPR-based therapies, yet its application is constrained by delivery challenges, including uncontrolled off-target activity and systemic genotoxicity. Thus, there is a need for a safe, efficient, and broadly applicable delivery platform for therapeutic gene editing.

### SUMMARY

The presently disclosed subject matter meets some or all of the above-identified limitations, as will become evident to those of ordinary skill in the art after a study of information provided in this document.

This summary describes several embodiments of the presently disclosed subject matter, and in many cases lists variations and permutations of these embodiments and implementations. This summary is merely exemplary of the numerous and varied embodiments and implementations. Mention of one or more representative features of a given embodiment or implementation is likewise exemplary. Such an embodiment or implementation can typically exist with or without the feature(s) mentioned; likewise, those features can be applied to other embodiments or implementations of the presently disclosed subject matter, whether listed in this summary or not. To avoid excessive repetition, this summary does not list or suggest all possible combinations of such features.

The present disclosure includes methods for producing engineered exosomes (eEXOs). In particular, embodiments of the present disclosure include methods for producing eEXOs in which: (a) a therapeutic payload is complexed with one or more magnetic nanoparticles (MNPs) to form a MNP-therapeutic payload complex; (b) the MNP-therapeutic payload complex is loaded into an endosome of a producer cell; (c) the endosome containing the MNP-therapeutic payload complex is extracted from the producer cell; and (d) the extracted endosome containing the MNP-therapeutic payload complex is extruded to produce the eEXO.

In some embodiments, the therapeutic payload is a clustered regularly interspersed short palindromic repeat (CRISPR) ribonucleoprotein (RNP) to facilitate the production of an eEXO which may be useful in gene editing applications. In some embodiments, the CRISPR RNP is selected from a CRISPR associated protein 9 (Cas9)-guide ribonucleic acid (gRNA) RNP, a base editor-gRNA RNP, and a prime editor-prime editing gene (PEG) RNA RNP. In some embodiments, the therapeutic payload is an antibody, such as IgG.

In some embodiments, the one or more MNPs have a core comprising magnetite. In some embodiments, the one or more MNPs are conjugated to one or more cell-penetrating peptides. In some embodiments, the one or more MNPs are conjugated to a poly-argenine peptide, a transactivating transcriptional activator (TAT) peptide, or a combination thereof. In some embodiments, the one or more MNPs have a core that is coated with 2-Distearoyl-sn-glycero-3-phosphoethanolamine-Poly(ethylene glycol) (DSPE-PEG).

In some embodiments, loading the MNP-therapeutic payload complex into the endosome includes applying a magnetic force to the MNP-therapeutic payload complex using one or more magnets. In some embodiments, extracting the endosome containing the MNP-therapeutic payload complex from the producer cell includes applying a magnetic force to the MNP-therapeutic payload complex using one or more magnets. In some embodiments, extracting the endosome containing the MNP-therapeutic payload complex from the producer cell includes homogenizing the producer cell.

In some embodiments, extruding the extracted endosome includes passing the extracted endosome through one or more porous membranes. In some embodiments, extruding the extracted endosome includes sequentially passing the extracted endosome through multiple membranes of decreasing size. In some embodiments, each membrane of the one or more porous membranes has a pore size ranging from about 200 nm to about 1000 nm.

In some embodiments, the method for eEXO production further includes conjugating a glycoprotein and/or fugogenic peptide to the engineered exosome.

The present disclosure further includes eEXOs. In certain embodiments, an eEXO includes: a therapeutic payload; one or more MNPs complexed to the therapeutic payload to form a MNP-therapeutic payload complex; and a vesicle containing the MNP-therapeutic payload complex. In some embodiments, the vesicle is derived from the endosomal membrane of an endosome from a producer cell in which the therapeutic payload was loaded.

Methods of treatment, gene editing, and tracking therapeutic payload delivery in a subject which make use of eEXOs produced in accordance with the present disclosure are also provided.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 is a schematic diagram showing an exemplary method for producing engineered exosomes (eEXOs) in accordance with an embodiment of the present disclosure, with portion “A” of the schematic diagram showing magnetic nanoparticle (MNP)-enriched endocytosis of a therapeutic payload (MF =magnetic force), portion “B” of the schematic diagram showing magnetic extraction of intracellular vesicles, and portion “C” of the schematic diagram showing generation of eEXOs through membrane extrusion.

FIG. 2A is a schematic diagram showing the modular synthesis of an MNP in accordance with an embodiment of the present disclosure, with portion “i” of the schematic diagram showing nanocrystal synthesis, portion “ii” of the schematic diagram showing encapsulation of the nanocrystal with 1,2-Distearoyl-sn-glycero-3-phosphoethanolamine-Poly(ethylene glycol) (DSPE-PEG), portion “iii” of the schematic diagram showing labeling of the encapsulated nanocrystal with lipophilic fluorophores, and portion “iv” of the schematic diagram showing surface conjugation with functional ligands.

FIG. 2B is a series of transmission electron microscopy (TEM) images of magnetite nanocrystals.

FIG. 2C is an image showing the gel shifting assay of maeleimide MNPs conjugated via cysteine-maleimide reaction with positively charged cysteine-modified transactivating transcriptional activator (TAT) peptides (amino acid sequence: CGGSYGRKKRRQRRR) at different ratios.

FIG. 3 is schematic diagram of a MNP made in accordance with an embodiment of the present disclosure.

FIG. 4A is a graph showing endocytotic uptake of magnetite MNPs enhanced by conjugation with cysteine-modified poly-arginine (Arg)₁₂ peptide and further enhanced by the application of a magnetic field, with the groups provided right of the vertical dashed line being incubated on a magnetic plate.

FIG. 4B is a series of images showing magnetite MNP-enhanced endocytosis of immunoglobulin G (IgG) using Bright field (top), Hoechst (middle), and IgG staining (bottom) (MF=magnetic field; Chl=chloroquine).

FIG. 5 is a schematic of a three-dimensionally printed cell homogenization device used to achieve consistent cell homogenization through shearing.

FIG. 6 is a schematic of a three-dimensionally printed device for extracting intracellular vesicles enriched from cell homogenate.

FIG. 7 is an image showing intracellular vesicles, including a mixture of endosomes and lysosomes containing MNPs, collected via magnetic extraction using a magnetic stand. MNPs conjugated with cysteine-modified (Arg)₁₂ peptide.

FIG. 8 is a series of images of cell suspension both before and after homogenization of the intracellular vesicles of FIG. 6 via shearing, with the homogenized vesicles containing MNPs and therapeutic cargos, as shown in the two bottom right panels. MNPs conjugated with (Arg)₁₂ peptides; cells of mouse macrophage cell line, RAW264.7. MNPs stained with DiI (FIG. 15B, #3). Model therapeutic cargo of IgG molecules labeled with fluorophores (Horse IgG, DyLight 488). Cell nuclei was stained with Hoechst 33342 dye.

FIG. 9A is a TEM image showing a vesicle acquired from a RAW 264.7 cell with MNPs with cysteine-modified (Arg)₁₂ peptides conjugated thereto provided in the vesicle.

FIG. 9B is a TEM image of eEXOs produced from vesicles like that of FIG. 9A.

FIG. 9C is a TEM image of natural exosomes produced by RAW 264.7 cells.

FIG. 9D is a graph showing the size distribution of control exosomes (natural exosomes from RAW 264.7 cells and eEXOs including MNPs with cysteine-modified (Arg)₁₂ peptides from RAW 264.7 cells.

FIG. 9E is a graph showing the size of control exosomes (natural exosomes from RAW 264.7 cells and eEXOs including MNPs with cysteine-modified (Arg)₁₂ peptides from RAW 265.7 cells. *p<0.05.

FIG. 9F is a graph showing the yield of control exosomes (natural exosomes from RAW 264.7 cells and eEXOs including MNPs with cysteine-modified (Arg)₁₂ peptides from RAW 265.7 cells. ***p<0.001.

FIG. 10 is a schematic showing magnetically enhanced endocytosis of a producer cell with MNP-ribonucleoprotein (RNP) complexes using an array of magnets.

FIG. 11 shows a simulated magnetic field generated by an array of neodymium magnets.

FIG. 12 is an image of the results of a Western blot assessing the abundance of endocytic markers RAB5A and lysosomal associated membrane protein 1(LAMP 1 ) on collected vesicles of FIG. 7 and revealing a time-dependent shift from early to late endosomes and lysosomes. WC =whole-cell lysate; CTL=vesicles extracted at 24 hours post-incubation. Cells incubated with MNP and therapeutic cargo for 30 minutes, 60 minutes, and 90 minutes. Fraction of vesicles can be controlled by varying the time of incubation (endocytosis).

FIG. 13 is an image of the results of gel electrophoresis showing MNP-RNP binding. Cas9 guide ribonucleic acid (gRNA)-RNPs targeting mouse β2M were incubated with MNPs at varying ratios and magnetically extracted; remaining supernatants tested with target DNA. Lane 1=no RNP; lane 2=RNP: MNP ratio of 1:0; lane 3=RNP: MNP ratio of 1:1; and lane 4=RNP: MNP ratio of 1:20.

FIG. 14 is an image showing the delivery rabies virus glycoprotein 29 (RVG29), or lack thereof, to the brains of a control mouse receiving phosphate-buffered saline (PBS) (leftmost positioned mouse in image) and mice receiving eEXOs modified with DSPE-PEG-RVG and labeled with lipophililic dye DiR (middle positioned and rightmost positioned mice in image) via tail vein injection 24 hours (h) post-injection. RVG29 linked to DSPE-PEG through conjugation by click chemistry.

FIG. 15A is a series of T2-weighted MRI images of MNPs of 6 nm to 40 nm dispersed in water.

FIG. 15B is an image showing 15 nm MNPs labeled with dicarbocyanine dyes. 1=MNP alone; 2=MNP/DiO; 3=MNP/DiI; 4=MNP/DiD; and 5=MNP/DiR.

FIG. 15C is a graph showing size dependence of T2 relaxivity of MNPs.

FIG. 15D is a fluorescence emission spectra of dye labeled MNPs shown in FIG. 19B scanned using a fluorometer.

FIG. 16A is a series of fluorescence images of RAW264.7 cells incubated with MNP-TAT on a magnetic plate.

FIG. 16B is a graph showing the uptake rate of MNP alone, MNP with conjugated TAT, MNP with an applied magnetic force; and MNP with conjugated TAT and an applied magnetic force. The uptake rate was calculated based on the iron content in the cells quantified using a colorimetric assay. ****p<0.0001.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the terms “peptide,” “polypeptide,” and “protein” are used interchangeably and refer to a compound comprising amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. Polypeptides can include protein complexes.

The terms “nucleotide”, “polynucleotide”, “nucleic acid” and “nucleic acid sequence” refer to deoxyribonucleotides or ribonucleotides and polymers thereof in either single or double stranded form unless stated otherwise or context precludes.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “subject” includes both human and animal subjects. Thus, veterinary therapeutic uses are provided in accordance with the presently disclosed subject matter. The term does not denote a particular age or sex.

As used herein, the terms “therapeutic payload” and “therapeutic cargo” are used interchangeably.

The presently disclosed subject matter is based, at least in part, on the discovery that magnetic nanoparticles (MNPs) can be utilized to both accelerate endocytosis of therapeutic cargos modeling (CRISPR) ribonucleotide protein (RNP) in producer cells and isolate cargo-enriched endosomes via magnetic extraction, and that the isolated, cargo-enriched endosomes can be extruded to produce engineered exosomes (eEXOs) of desired size. Accordingly, in one aspect, the present disclosure includes methods for producing an engineered exosome useful in gene editing applications.

A method for producing an engineered exosome commences with complexing a therapeutic payload with one or more MNPs to form a MNP-therapeutic payload complex. Therapeutic payloads which may be complexed with MNPs in various embodiments include peptides, nucleotides, combinations thereof, and/or other biomolecules that may be useful in the treatment of a subject. In some embodiments, a CRISPR RNP is complexed with one or more MNPs to form a MNP-RNP complex. As will become further evidenced by the discussion that follows, the loading strategy employed in the disclosed production methods is believed to enable the loading of all forms of CRISPR RNPs. In this regard, binding between MNPs and RNPs can be achieved via electrostatic interaction. RNPs are negatively charged, while MNPs can be conjugated with peptides to exhibit a positive charge. Accordingly, binding of negatively charged CRISPR RNPs and positively charged MNPs can be achieved by mixing the two in a neutral pH solution. In some embodiments, the CRISPR RNP is selected from a CRISPR associated protein 9 (Cas9)-guide ribonucleic acid (gRNA) RNP, a base editor-gRNA RNP, and a prime editor-prime editing gene (PEG) RNA RNP. Accordingly, in various embodiments, the CRISPR RNP can include Cas9 or variants thereof, such as Cas9 nickase or inactive Cas9, alone or in combination with other enzymes, such as a deaminase or a reverse transcriptase. Depending on the CRISPR RNP utilized, the manner by which gene editing occurs will vary. In this regard, and in various embodiments, the CRISPR RNP can facilitate deoxyribonucleic acid (DNA) modification through double-stranded break, base conversion, or reverse transcription-mediated rewriting to accommodate various gene editing applications. In various embodiments where the CRISPR RNP is a base editor-gRNA RNP, the base editor-gRNA RNP may be a cytosine base editor (CBE) or an adenine base editor (ABE). The particular gRNA sequence utilized in the CRISPR RNP can be identified based on the desired target for gene editing and using techniques and tools known in the art.

MNPs that can be utilized include those with a core shell-structure. In some embodiments, the MNPs utilized include an iron oxide core, such as magnetite (Fe₃O₄) or maghemite (Fe₂O₃), that is coated. Coatings which may be applied to the core of the MNPs include those which facilitate binding to RNPs as well as those which enable detection or tracking of the MNPs and the therapeutic cargo with which it is bound. In this regard, coatings which can be applied to the MNPs include, by way of non-limiting example, fluorophores, DSPE-PEG, or a combination thereof. The core size of the MNPs can be selected to limit the extent to which the MNPs decrease the surface-area-to-volume ratio of the therapeutic payload. In some embodiments, the core of the MNPs exhibit a size ranging from about 10 nm to about 20 nm. In some embodiments, the core of the MNPs is about 10 nm, about 15 nm, and/or about 20 nm. In some embodiments, the core is in the form of metal nanocrystals. In this regard, magnetite nanocrystals can be synthesized via thermal decomposition using known techniques and methods. Nanocrystals resulting from such process may, however, be hydrophobic. In such cases, coating the nanocrystal core with DSPE-PEG can serve to improve coating uniformity and facilitate precise surface modification that aids in MNP-cargo binding.

MNPs can enhance endocytotic uptake of therapeutic payloads and enable magnetic purification. Cell-penetrating peptides and magnetofection further promote endocytosis, while trafficking inhibitors such as chloroquine minimize lysosomal degradation. Together, these features improve loading efficiency, reproducibility, and scalability compared with conventional methods. Cell-penetrating peptides can also facilitate MNP binding with the therapeutic payload and enhanced cellular uptake by producer cells. Accordingly, in some embodiments, the MNPs complexed with the therapeutic payload are conjugated to one or more cell-penetrating peptides.

Cell-penetrating peptides that can be conjugated to the MNPs include, by way of non-limiting example, transactivating transcriptional activator (TAT) peptide, poly-arginine peptide, such as poly-arginine 12 ((Arg)₁₂), and combinations thereof. The cell-penetrating peptides utilized can, of course, vary depending on the application and/or environment in which the MNPs will be used. For instance, the cell-penetrating peptide utilized can vary depending on the therapeutic payload with which the MNPs are complexed. Tools, such as the CPPsite 2.0 Database of Cell-Penetrating Peptides available at http://crdd.osdd.net/raghava/cppsite/, and techniques for identifying cell-penetrating peptide candidates for particular applications are known in the art and readily available. In instances where a peptide is conjugated to a MNP, the MNP can be characterized as including such peptide.

It is appreciated that while the core of the MNPs is sometimes referred to as being comprised of iron oxide, that MNPs of alternative material composition can also be utilized without departing from the spirit and scope of the present disclosure. As such, embodiments in which alternative metal nanoparticles, including by way of non-limiting example, cobalt, manganese, nickel, iron, and iron oxide doped with zinc, nickel, cobalt, or manganese nanoparticles, are also contemplated herein.

As noted, complexing of the therapeutic payload to the MNPs can be achieved through electrostatic interaction between the therapeutic payload and the MNPs. In this regard, therapeutic payloads such as IgG antibodies and CRISPR-RNPs are negatively charged at physiological pH levels, and the MNPs can be synthesized or functionalized to exhibit a positive charge. In some embodiments, the MNPs are functionalized to exhibit a positive charge by applying one or more positively charged ligands to the MNPs. In various embodiments, the MNPs can be functionalized with poly-arginine peptides, TAT peptides, or combinations thereof to cause the MNPs to exhibit a positive charge and promote electrostatic binding with the therapeutic payload. In the case of therapeutic payloads with RNPs, such as CRISPR RNPs, binding of the MNPs to the therapeutic payload can also be achieved using, by way of non-limiting example, coiled-coil peptides, spytag-spycatcher peptides, biotin-avidin, anti-cas9 antibody.

Once the therapeutic payload is complexed with the MNPs, in the production method, the MNP-therapeutic payload complex is then loaded into an endosome of a producer cell. Loading of the MNP-therapeutic complex can be achieved via endogenous loading by leveraging the natural endocytic pathways of the producer cell to thereby bypass the traditional sequence of production, collection, and post hoc loading in conventional exosome production methods and thus avoiding or limiting the low production yields, inefficient cargo loading, and/or batch-to-batch variability associated with the same. To promote endogenous loading, the producer cell can be incubated with the MNP-therapeutic payload complex in standard cell culture conditions, except for the removal of serum from the culture to prevent serum proteins from interfering with complex binding. In some embodiments, endogenous loading may be facilitated by incubating the producer cell with the MNP-therapeutic payload complex in a carbon dioxide incubator for 90 minutes.

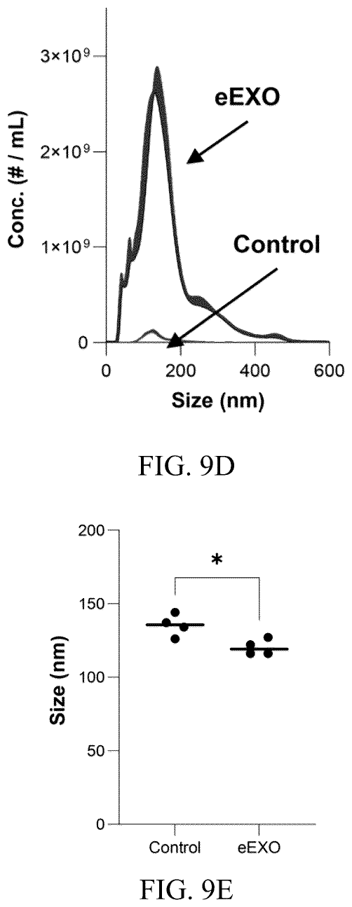

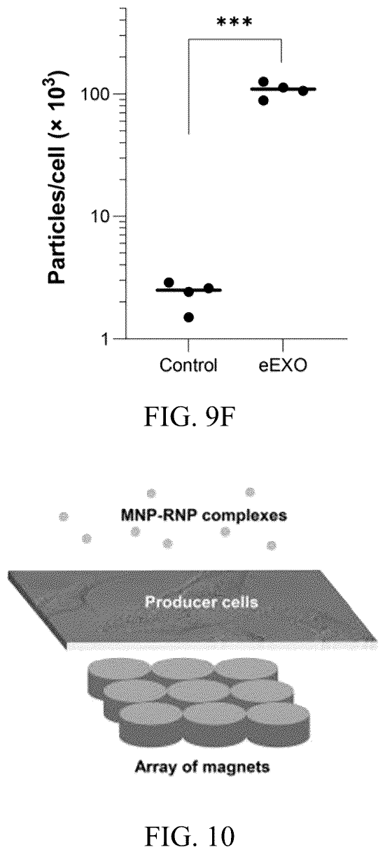

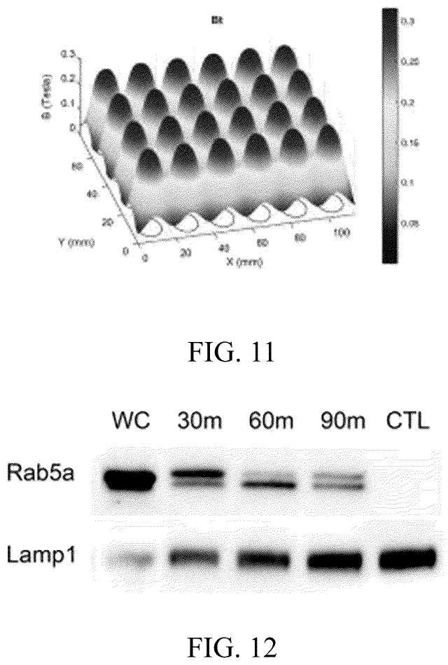

It has been discovered that MNP endocytosis can be enhanced through the application of magnetic force. Accordingly, in some embodiments, loading of the MNP-therapeutic payload complex includes applying a magnetic force to the MNP-therapeutic payload complex via one or more magnets. In some embodiments, multiple magnets are utilized to apply the magnetic force. In this regard, the producer cell and the MNP-therapeutic payload complex can be incubated on a magnetic plate assembled with magnets. Magnet type, geometry, and/or plate thickness can be optimized to maximize magnetic pulling force to maximize accumulation of the MNP-therapeutic payload complex in the endosomal compartment. It has been found that an array of neodymium magnets (FIGS. 9 and 10) are particularly useful for loading applications. In this regard, neodymium magnets are the strongest permanent magnets and are available in many shapes. Of course, other types of magnets can also be utilized, although the magnetic enhancement may vary based on the field design.

To enhance loading retention and prevent lysosomal degradation, endosomal-lysosomal trafficking inhibitors, such as chloroquine, nocodazole, and CID1067700, which modulate endosomal pH, disrupt cytoskeletal dynamics, or inhibit endosomal-lysosomal fusion can optionally be evaluated. Subcellular distribution of purified vesicles along the endocytic pathway can be assessed via western blotting for specific markers, including Rab5 (early endosome) and Lamp1 (late endosome/lysosome) among others (FIG. 12). The nature of magnetically collected vesicles can be controlled by varying the time of incubation for endocytotic loading.

The production methods disclosed herein can be utilized to generate sufficient amounts of engineered exosomes for a variety of cell types, including primary cells, stem cells, immortalized cell lines, and other cells that can be attached to a culture dish. Accordingly, in various embodiments, producer cells utilized in the loading of the MNP-therapeutic cargo complex can include, by way of non-limiting example, blood cells (e.g., peripheral blood mononuclear cells (PBMCs)), bone marrow derived stromal cells (BMDSCs), macrophage or marcrophage-like cells (e.g., RAW264.7 cells), osteolineage cells, and endothelial cells. In clinical use applications, the disclosed production methods can allow the generation of engineered exosomes from patient cells. The use of exosomes derived from patient cells can be particularly advantageous as it can minimize adverse patient immune responses and exosomes from different cell types can exhibit different tropism, i.e., target particular cell types in the body. Accordingly, in some embodiments of the disclosed production methods, the producer cells are obtained from a subject that is the target of treatment or administration of the eEXO. In this way, the production methods of the present disclosure may facilitate the production of autologous eEXOs that reduce immunogenicity issues and support repeated dosing to a subject.

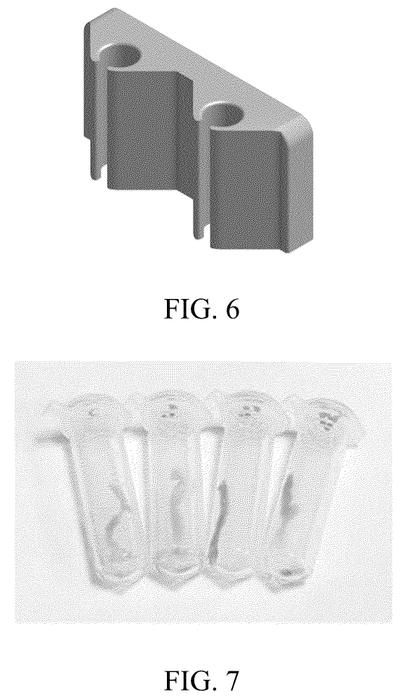

After the MNP-therapeutic cargo is loaded into the endosome, in the production method, the intracellular endosome enriched with the MNP-therapeutic cargo is subsequently extracted from the producer cell. To facilitate extraction of the enriched endosome from the producer cell, the producer cell can be homogenized utilizing suitable homogenization techniques. Techniques which may be employed for cell homogenization include, by way of non-limiting example probe sonication, bath sonication, and mechanical shearing. Mechanical shearing has surprisingly been found to be particularly effective with respect to facilitating the release of intracellular vesicles while preserving nuclear integrity and avoiding the inclusion of DNA in the extracted endosome. In some embodiments, mechanical shearing is achieved by passing the producer cell containing the enriched endosome through a 30 gauge (30G) needle. To ensure batch-to-batch consistency, a device configured to standardize the shearing process can be employed, such as that shown in FIG. 5, which was designed to fix the collection tube and shearing syringe in place. Following homogenization of the producer cell with the endosome enriched with the MNP-therapeutic payload complex, the enriched endosome is isolated from cell homogenate. In this regard, the presence of the MNPs of the MNP-therapeutic payload complex can be leveraged by applying a magnetic force to magnetically extract the enriched endosome from the cell homogenate. In some embodiments, extraction of the enriched endosome from cell homogenate is achieved using a dedicated extraction device configured to apply a magnetic force, such as that shown in FIG. 6. The device was developed using three-dimensional printing and includes two tubes, with a neodymium magnet (1″×¼″×¼″) attached to the side of each tube.

Following isolation the enriched endosome, in the production method, the enriched endosome is extruded to produce the eEXO. Extrusion of preloaded intracellular vesicles ensures that most resulting engineered exosomes will contain the MNP-therapeutic payload complex. In this regard, the disclosed production methods are believed to be more efficient than conventional methods that collect the exosome first and then load the therapeutic payload into the exosome. Following extrusion, the MNP-therapeutic payload complex is contained in a vesicle corresponding to the membrane of the enriched endosome. In some embodiments, extrusion of the enriched endosome is achieved by passing the enriched endosome through one or more porous membranes. The pore size of the membrane or membranes can be adjusted so that the eEXO produced is of a desired size. In some embodiments, extrusion of the enriched endosome includes sequentially passing the enriched endosome through multiple membranes of decreasing pore size. In some embodiments, each membrane of the multiple membranes has a pore size ranging from about 1000 nm to about 200 nm. Multi-step extrusion can help to reduce resistance during extrusion. In some embodiments, the enriched endosome is passed through a membrane of a given size multiple times. In this regard, it has been surprisingly found that after passing a MNP-therapeutic payload enriched endosome through a 200 nm pore-sized membrane ten times that the size of the eEXO closely approximates that of natural exosomes obtained through conventional ultracentrifugation. In exome embodiments, the eEXO produced may have a size of about 100 nm.

In some embodiments, the production method can further include conjugating one or more targeting ligands to the produced eEXO to enhance or otherwise alter performance and/or delivery of the eEXO within a subject. In this regard, blood-brain barrier (BBB) penetration may be improved through the conjugation of certain peptides. Among such peptides is the rabies virus glycoprotein fragment RVG29 (RVG29), which is well established to bind to nicotinic acetylcholine receptors on brain microvascular endothelium, mediating receptor-dependent endocytosis. RVG29 also supports retrograde axonal transport and tarns-synaptic spread, which can further promote distribution within the CNS of a subject. Accordingly, in some embodiments, the method may further include conjugating the produced eEXO with RVG29. To facilitate conjugation of RVG29 to the eEXO, RVG29 may be linked to DSPE-PEG copolymer via click chemistry. In some embodiments, one or more fugogenic peptides is conjugated to the eEXO to limit the extent to which CRISPR RNP, or other applicable therapeutic payload, is limited as a result of endosomal entrapment. In this regard, and in some embodiments, INF7 peptide (amino acid sequence: GLFEAIEGFIENGWEGMIDGWYG) is conjugated to the eEXO.

In instances where ligands or other molecules are conjugated to the eEXO, the eEXO can be characterized as including such ligands or other molecules.

Although the above production methods are generally described above in the context of producing a single eEXO, it is appreciated that such production methods can likewise be employed in the production of multiple eEXOs. Indeed, multiple therapeutic payloads and producer cells can be utilized to simultaneously produce large quantities of eEXOs.

In another aspect, the present disclosure includes engineered exosomes (eEXOs), such as those which may be produced via the various production methods disclosed herein. An artificially engineered exosome made in accordance with the present disclosure includes: a therapeutic payload; one or more MNPs complexed to the therapeutic payload, such that the MNPs and the therapeutic payload form a MNP-therapeutic payload complex; and a vesicle containing the MNP-therapeutic payload complex.

In some embodiments, the therapeutic payload of the eEXO is an antibody, such as IgG. In some embodiments, the therapeutic payload is a CRISPR RNP. In various embodiments, the CRISPR RNP is a Cas9-gRNA RNP, a base editor-gRNA RNP, or a prime editor-PEG RNA RNP. In some embodiments, the base editor-gRNA RNP is a cytosine base editor. In some embodiments, the base editor-gRNA RNP is an adenine base editor.

In some embodiments, the MNPs of the eEXO include an iron oxide core. In some embodiments the iron oxide core of the MNPs is magnetite or maghemite. In some embodiments, the core of the MNPs is selected from cobalt, manganese, nickel, iron, and iron oxide doped with zinc, nickel, cobalt, or manganese nanoparticles. In some embodiments, the core of the MNPs is coated with fluorophores, DSPE-PEG, or a combination thereof. In some embodiments, the core of the MNPs is in the form of metal nanocrystals. In some embodiments, the MNPs include a magnetite core that is coated with DSPE-PEG. In some embodiments, the MNPs include one or more cell-penetrating peptides. In some embodiments, the one or more cell-penetrating peptides include one or more poly-arginine peptides, such as Arg₁₂ peptides, one or more TAT peptides, or combinations thereof. In some embodiments, the MNPs exhibit a size ranging from about 10 nm to about 20 nm. In some embodiments, the MNPs exhibit a size of about 10 nm, about 15 nm, and/or about 20 nm.

In some embodiments, the vesicle of the eEXO corresponds to a portion of the endosomal membrane of an endosome of a producer cell. In various embodiments, the producer cell from which the endosome, and thus portion of the endosomal membrane was derived may be a blood cell (e.g., a peripheral blood mononuclear cell (PBMC)), a bone marrow derived stromal cell (BMDSC), a macrophage or marcrophage-like cell (e.g., RAW264.7 cell), an osteolineage cell, and endothelial cells.

In some embodiments, the artificially engineered endosome includes one or more ligands configured to enhance or otherwise alter the performance and/or delivery of the eEXO conjugated to the vesicle of the eEXO. In some embodiments, RVG29 is conjugated to the vesicle of the eEXO to improve BBB penetration of the eEXO. In some embodiments, the RVG29 is linked to DSPE-PEG copolymer. In some embodiments, a fugogenic peptide is conjugated to the vesicle of the eEXO to limit the extent to which CRISPR RNP, or other applicable therapeutic payload, is limited as a result of endosomal entrapment. In some embodiments, the fugogenic peptide is INF7 peptide.

The eEXOs can be produced to have a diameter that mimics that of natural exosomes. In this regard, and in some embodiments, the produced eEXOs have a diameter ranging from about 100 nm to about 150 nm. Of course, eEXOs with other diameters can also be produced by utilizing membranes with different pore sizes during the extrusion process.

eEXOs produced in accordance with the present disclosure may find utility in a variety of applications in which delivery of a therapeutic payload to a subject is needed. Accordingly, in another aspect, the present disclosure includes methods of treatment that include administering one or more eEXOs disclosed or produced in accordance with the present disclosure to a subject in need thereof. The therapeutic payload of the eEXOs can be selected based on the disease or ailment intended for treatment. eEXOs produced in accordance with the present disclosure can be administered to a subject using any suitable administration method or route that facilitates desired transport of the eEXOs and delivery of the therapeutic payload in the manner necessary to treat the subject. For instance, in various embodiments, administration methods which may be utilized include, but are not limited to, systemic administration, parenteral administration (including intravascular, intraosseous, intracerebroventricular, intrathecal, intramuscular, and/or intraarterial administration), oral delivery, subcutaneous administration, intraperitoneal administration, dermally (e.g., topical application), and local injection.

CRISPR RNPs, such as Cas9-gRNA RNPs, can mediate immediate gene-editing without requiring transcription or translation in target cells. Furthermore, the short intracellular half-life of these RNPs can minimize off-target editing compared to DNA- or RNA-based systems. Compared to synthetic materials or viral vectors, exosomes offer improved biocompatibility, reduced immunogenicity, and the improved ability to cross biological barriers. Exosomes also exhibit inherent cell-type-specific tropism, making them attractive delivery vehicles for diverse therapeutic payloads. These features make exosomes uniquely suited for safe and efficient delivery of CRISPR tools. The eEXOs and methods for eEXO production disclosed herein may thus prove particularly useful in CRISPR-based gene-editing applications. Accordingly, in some embodiments the therapeutic payload of the eEXO or eEXOs administered to a subject include CRISPR RNP to facilitate gene editing in the subject.

MNPs are highly sensitive magnetic resonance imaging (MRI) contrast agents. MNPs encapsulated in eEXOs can offer image guidance to evaluate the distribution of the therapeutic payloads, such as CRISPR RNPs, in the body and facilitate optimization of treatment schemes. Accordingly, in a further aspect, the present disclosure includes methods for tracking the delivery of a therapeutic payload, such as a CRISPR RNP or antibody to a subject, which include administering one or more eEXOs disclosed or produced in accordance with the present disclosure to a subject and subsequently imaging the subject or a biopsy acquired from the subject with MRI to acquire one or more images of the subject or biopsy. The one or more images can then be analyzed to detect the presence of the one or more eEXOs in the one or more images. In some embodiments, the one or more images are of a brain of the subject.

The concentration of MNPs can also be accurately measured using colorimetric assays. Accordingly, embodiments, in which one or more eEXOs disclosed or produced in accordance with the present disclosure are administered to a subject and a colorimetric assay of a biological sample obtained from the subject is subsequently performed to track delivery of a therapeutic payload in a subject. Accordingly, the MNPs of the eEXOs disclosed or produced in accordance with the present disclosure can enable the assessment of eEXO biodistribution at various spatial scales.

Characterization of eEXOs can be performed according to the minimum requirements for the reproducible and accurate investigation of exosomes proposed by the International Society for Extracellular Vesicles (ISEV).

The presently disclosed subject matter is further illustrated by the following specific but non-limiting examples. Some of the following examples may include aspects that are prophetic, notwithstanding the numerical values, results and/or data referred to and contained in the examples. Further, the following examples may include compilations of data that are representative of data gathered at various times during the course of development and experimentation related to the present invention.

### EXAMPLE

### Engineered Exosome (eEXO) Production Utilizing Magnetic Nanoparticles (MNPs)

By combining Cas nucleases with programmable guide RNAs (gRNAs), CRISPR-based gene editing systems enable precise deletion, insertion, modification or regulation of genomic loci. Compared with conventional small-molecule or biologic drugs, CRISPR offers several distinct advantages, including rational design, rapid turnaround, and multiplexed editing capacity. These unique features, when paired with advances in clinical sequencing, open the door to personalized gene editing therapies. However, current clinical approaches rely on ex vivo editing of hematopoietic stem/progenitor cells (HSPCs) with reinfusion after myeloablative conditioning. Although effective, this process is costly, invasive, and limited to specialized centers, making it applicable to only a small subset of patients. In vivo editing has the potential to reduce cost, eliminate the need for intensive conditioning regimens, and broaden the scope of gene editing therapies across diverse diseases.

Despite its promise, in vivo CRISPR delivery carries risks. gRNAs can tolerate mismatches, and Cas nucleases may bind to sequences with mismatches or bulges, leading to off-target editing and potentially tumorigenic mutations. Systemic delivery further amplifies concerns about prolonged exposure and genotoxicity. Viral vectors, though widely used, distribute broadly after systemic administration and can drive long-term transgene expression through genomic integration or episomal presence, raising additional safety concerns. Moreover, the clinical utility of viral vectors such as adeno-associated virus (AAV) is limited by widespread pre-existing immunity. Additionally, the single stranded DNA delivered by AAVs can activate the body's viral defense system through pathogen-associated molecular patterns (PAMP) receptors, leading to immune-related adverse effects, including edema. Furthermore, viral vectors can induce long-term Cas9 expression by integrating into the host genome or maintaining stable extrachromosomal expression, significantly increasing the risk of off-target gene editing. Lipid nanoparticles offer a non-integrating alternative, but Cas9 activity in quiescent cells, such as long-term HSPCs, is constrained by low transcriptional activities. As a result, ongoing clinical trials in therapeutic gene editing remain largely confined to localized delivery sites (e.g., subretinal space) or to ex vivo manipulation of isolated cells.

Exosomes are nanoscale vesicles released by virtually all cell types and serve as natural carriers of bioactive molecules such as lipids, nucleic acids, and cytokines, mediating intercellular communication across tissues. Natural exosomes are produced through the endosomal pathway. Briefly, late endosomes undergo inward membrane invagination to form multivesicular bodies (MVBs) filled with intraluminal vesicles (ILVs). When MVBs fuse with the plasma membrane, ILVs are released as exosomes, inheriting the lipid membrane and surface markers of their endosomal origin. Exosome output is intrinsically constrained by the need for cellular homeostasis. However, it has been found that upregulating genes involved in exosome biogenesis and metabolism can increase production by 15 to 40 fold, suggesting that under normal conditions only a small fraction of endosomal membranes are directed toward exosome release.

Unlike synthetic materials or viral vectors, exosomes possess remarkable immune compatibility and are less susceptible to being intercepted by the mononuclear phagocytic system (MPS), thanks to the display of self-antigens on their surface. Notably, exosomes can effectively traverse the blood-brain barrier (BBB), offering promise as delivery vehicles for therapeutics targeting brain-related diseases. Previous research has elucidated that exosomes traverse the BBB via the transcytosis pathway, involving their endocytosis at the luminal surface of brain microvascular endothelium, followed by release at the abluminal side.

As endogenous carriers, exosomes offer superior biocompatibility and reduced immunogenicity compared to synthetic nanoparticles or viral vectors. Preclinical and clinical studies have shown that engineered exosomes (eEXOs) can cross biological barriers such as the blood brain barrier (BBB) and dense tumor stroma, underscoring their translational potential for delivering therapeutic agents including gene editing tools.

Despite their promise, the therapeutic development of exosomes is limited by several interrelated challenges, most notably low production yields, inefficient cargo loading, and high batch-to-batch variability. Current methods rely on a sequential process of exosome biogenesis, collection, and post hoc cargo loading. This approach is both labor-intensive and inefficient. In this regard, production yields remain low, with conventional techniques producing only thousands of particles per cell, necessitating large bioreactors for cell culture and costly purification steps. Known cargo loading methodologies rely on membrane-permeabilization techniques, such as electroporation, sonication, freeze-thaw cycling, or pH-gradient modulation, which provide only modest encapsulation efficiency. In cases involving small-molecule therapeutic agents, like siRNA, low loading efficiency can be mitigated by mixing exosomes with highly concentrated molecules. However, for larger molecules such as Cas9-gRNA RNPs, this approach is economically prohibitive, and the subsequent purification process is exceedingly challenging. In other words, these limitations on cargo loading are generally prohibitive in applications involving large cargos such as Cas9-gRNA RNPs. Furthermore, the multistep production process further contributes to inconsistency, which contributes to high batch-to-batch variability. Safety concerns also persist. Repeated dosing of allogeneic exosomes risks immune rejection due to MHC mismatch, while exosomes from immortalized cells may carry oncogenic material, raising the possibility of promoting malignancy in non-terminal disease settings.

To date, many synthetic biology and nanofabrication strategies have focused on optimizing individual steps of producing cargo-loaded exosomes. For example, it has been shown that genetically modified producer cells can enhance exosome yields and improves the loading of specific cargos such as mRNA or Cas9 protein. Other approaches have used mechanical homogenization to generate extracellular vesicle-mimicking particles, and it has been shown that various cell types upregulate exosome production in response to stress. Although membrane permeabilization and protein fusion techniques have been developed to boost cargo incorporation, these methods are often limited to specific cell types and/or constrained by narrow applications.

Hence, the inventors of the present application set out to develop a novel strategy for producing engineered exosomes (eEXOs) that facilitates the loading and transport of CRISPR RNPs and bypasses the traditional sequence of exosome production, collection, and post hoc loading.

### Strategy for Exosome Production

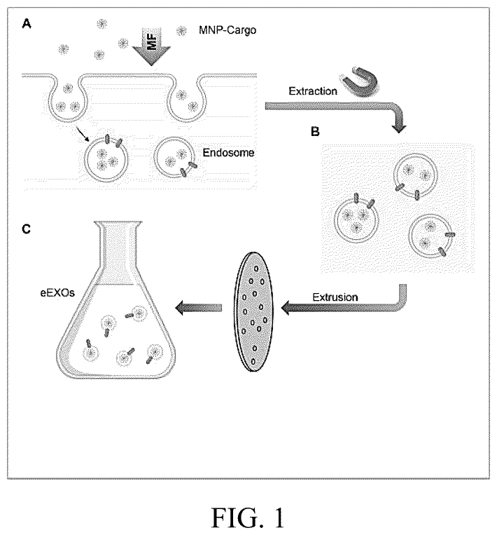

The strategy for exosome production underlying the present example leverages natural endocytic pathways to exploit endosomes as the starting material for eEXO production. As shown in FIG. 1, the strategy utilizes magnetic nanoparticles (MNPs) to initially accelerate the endocytosis of therapeutic cargos and then subsequently isolate cargo-enriched endosomes via magnetic extraction. The isolated, cargo-enriched endosomes are then extruded to produce eEXOs of a controlled size (FIG. 1). Without wishing to be bound by any particular theory, it is believed that this strategy maximizes utilization of preloaded endosomes, preserves the native membrane composition of exosomes, and reduce contamination from intracellular debris. Importantly, preloading ensures that most eEXOs carry therapeutic cargos, dramatically improving efficiency. This strategy, and again without wishing to be bound by any particular theory, is believed to be broadly applicable across multiple cell types, including patient-derived cells, enabling autologous eEXO production that reduces immunogenicity and supports repeat dosing. The disclosed strategy is further believed to be ideal for large-scale production of eEXOs as it eliminates two critical bottleneck steps: exosome collection through ultracentrifugation of a large volume of culture media, and cargo loading via membrane permeabilization.

### Synthesis of MNPs

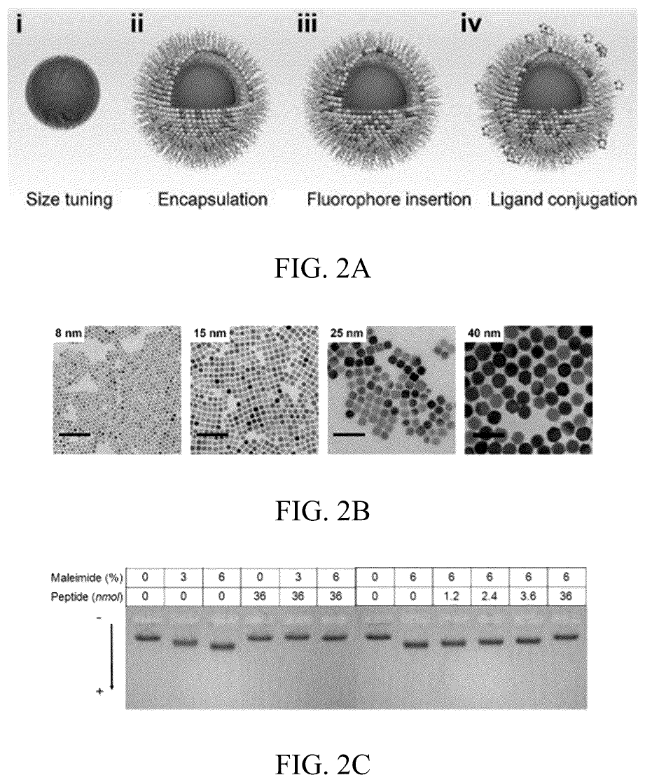

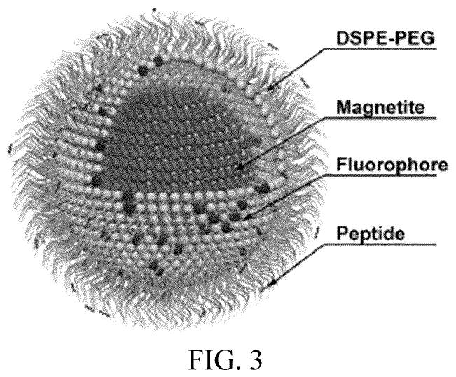

In the studies underlying the disclosed example, MNPs with a core-shell structure, including magnetite (Fe₃O₄) nanocrystals coated with DSPE-PEG were utilized. DSPE-PEG is a polymer utilized in several therapeutics approved by the United States Federal Drug Administration. The crystal core determines the magnetic properties, while the polymer coating confers water dispersity and provides sites for functionalization. In this regard, a previously developed synthesis platform enabling modular design of the core, coating, and surface ligands was used in the synthesis MNPs (FIGS. 2A and 3). See Zhang LL, et al., “Lipid-Encapsulated Fe3O4 Nanoparticles for Multimodal Magnetic Resonance/Fluorescence Imaging”, ACS Applied Nano Materials. 2020; 3(7):6785-97.

Magnetite nanocrystals can be synthesized via thermal decomposition utilizing known techniques (FIG. 2B). See Tong S., et al., “Size-Dependent Heating of Magnetic Iron Oxide Nanoparticles”, ACS Nano. 2017; 11(7):6808-16. The resulting nanocrystals are, however, hydrophobic and dispersible only in nonpolar solvents. To overcome this, a dual solvent-exchange (DSE) method has been developed to coat the nanocrystals with DSPE-PEG, a copolymer widely used in liposomal drugs and mRNA vaccines. See Tong S., et al., “Self-assembly of phospholipid-PEG coating on nanoparticles through dual solvent exchange”, Nano Lett. 2011; 11(9):3720-26.

A study was performed to assess the extent to which the DSE method improves coating uniformity over conventional approaches. In the study, MNPs were generated using the method described in Zhang LL, et al., “Lipid-Encapsulated Fe3O4 Nanoparticles for Multimodal Magnetic Resonance/Fluorescence Imaging”, ACS Applied Nano Materials. 2020; 3(7):6785-97. MNPs were coated with DSPE-PEG. DSPE-PEG maleimide was added to the coating materials at different ratios indicated in FIG. 2C. MNPs were mixed with cysteine-TAT peptides at different concentration. The difference of peptide density on MNPs were evaluated by gel electrophoresis. The MNPs with more TAT peptides had more positive charge and ran further to the bottom during electrophoresis. This demonstrated great control over peptide conjugation, which is very difficult using other synthesis methods. The study showed that the DSE method markedly improves coating uniformity over conventional approaches. Moreover, this approach enables precise functionalization of MNPs with various reactive groups, providing control over ligand conjugation. As shown with a gel shifting assay (FIG. 2C), the density of TAT peptides conjugated onto the coated MNP surface can be tightly regulated, which presents a pivotal step in optimizing the binding interactions between MNPs and Cas9-gRNA RNPs and controlling the endocytosis of MNPs. Importantly, it also enables precise surface modification, which is essential for enhancing MNP-cargo binding and promoting endocytosis (FIG. 2C).

### MNP as a Multimodality Imaging Contrast Agent

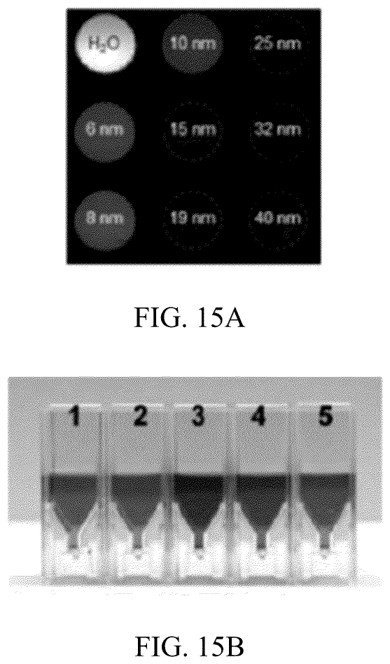

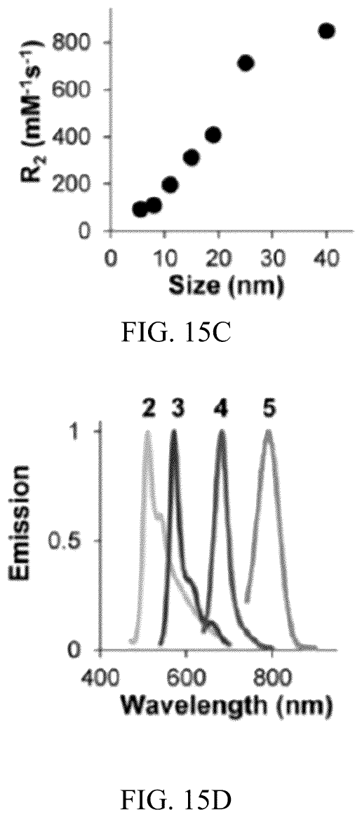

Studies have shown that DSPE-PEG coated MNPs have higher MRI T2 relaxivity compared with Feridex I.V.® or Resovist®. T2 relaxivity increases with the core size from 6 nm to 40 nm (FIGS. 15A and 15C). A method for labeling MNPs with various long acyl chain dicarbocyanine dyes widely used for membrane staining (FIG. 15B) was developed. FIG. 15D shows the emission spectra of the MNPs labeled with four different dicarbocyanine dyes (DiO, DiL, DiD, and DiR) with emission peaks from 505 nm to 780 nm. The versatile dye labeling technique and the intrinsic T2 relaxivity of MNPs enable optimizing cellular uptake by producer cells, evaluating cargo loading efficiency in eEXOs, tracking the biodistribution of eEXOs, and assessing the distribution of eEXOs among different cell populations in the brain using a combination of fluorescence microscopy, near infrared live animal imaging, and magnetic resonance imaging.

### Cellular Uptake of Therapeutic Cargos

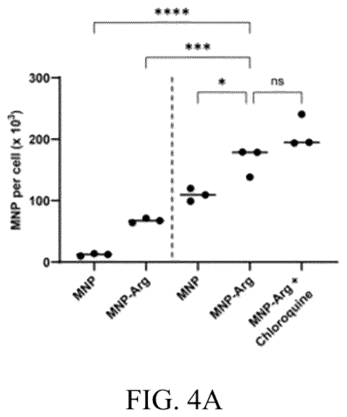

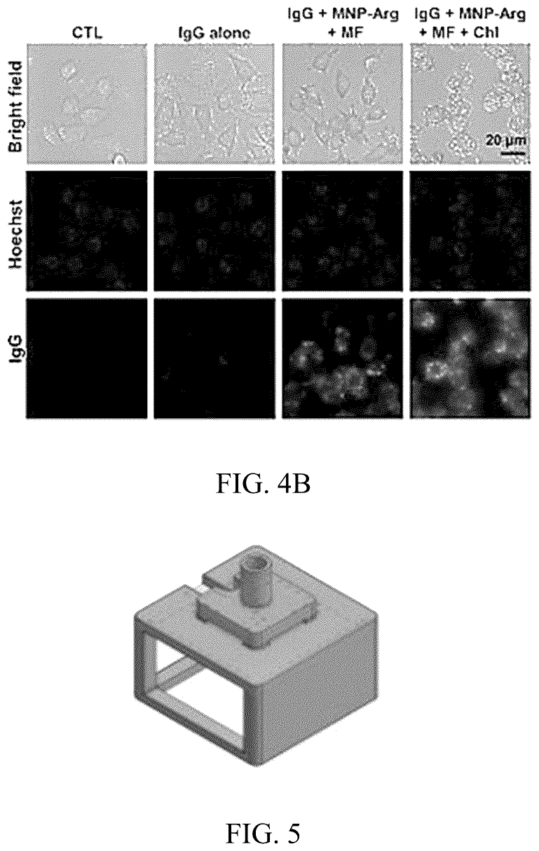

It has been found that MNP endocytosis can be enhanced across various cell types using cell-penetrating peptides and magnetic induction. A study was performed to evaluate whether MNPs could similarly improve cellular uptake of therapeutic agents. In the study, magnetite MNP conjugated with arginine peptides were mixed with the model cargo described below in a serum-free medium. The cells were incubated with the medium for 90 minutes. After incubation, the cells were washed and harvested and the amount of MNPs in the cells were quantified using a Ferrozine assay (FIG. 4A). Cells were fixed with paraformaldehyde staining with Hoechst 33342 and imaged with a Nikon fluorescence microscope (FIG. 4B).

Because CRISPR RNP cargos are negatively charged at physiological pH, MNPs functionalized with poly-arginine peptides, (Arg)₁₂, were engineered to promote electrostatic binding and endocytosis. Conjugation with (Arg)₁₂ plus magnetic induction increased MNP uptake >10-fold (FIG. 4A). Notably, chloroquine, an inhibitor of endosomal-lysosomal trafficking, did not reduce uptake. Chloroquine was used to control the transport of cargos from endosome to lysosome inside the cells. Natural exosomes are generated from late endosomes and multivesicle bodies. Chloroquine was used to retain MNP-cargo complex in the endosomes so that the final engineered exosomes would have a similar membrane source as that of natural exosomes.

To test macromolecular cargo loading, fluorescently labeled IgG was used as a model cargo for Cas 9-gRNA RNPs. IgG has a molecular weight of 150 kD and a zeta potential of −16.1 mV, similar to that of Cas9-gRNA RNPs, with Cas9 nuclease being a large protein of approximately 160 kDa. IgG alone showed minimal uptake by RAW 264.7 cells, whereas MNP-Arg complexes markedly enhanced internalization under magnetic induction (FIG. 4B). Cells treated with chloroquine exhibited enlarged endosomes containing IgG, consistent with blocked lysosomal maturation. These findings demonstrate that multifunctional MNPs overcome charge-related barriers and significantly enhance uptake of therapeutic cargos. In view of the fact that poly (Arg)ₙ peptides have previously been utilized for SiRNA delivery and the preliminary data showing that MNPs conjugated with (Arg)₁₂ can bind to IgG molecules (i.e., the model cargo for Cas9-gRNA RNPs), it is believed that similarly functionalized MNPs can be utilized to generate eEXOs carrying Cas9-gRNA RNPs.

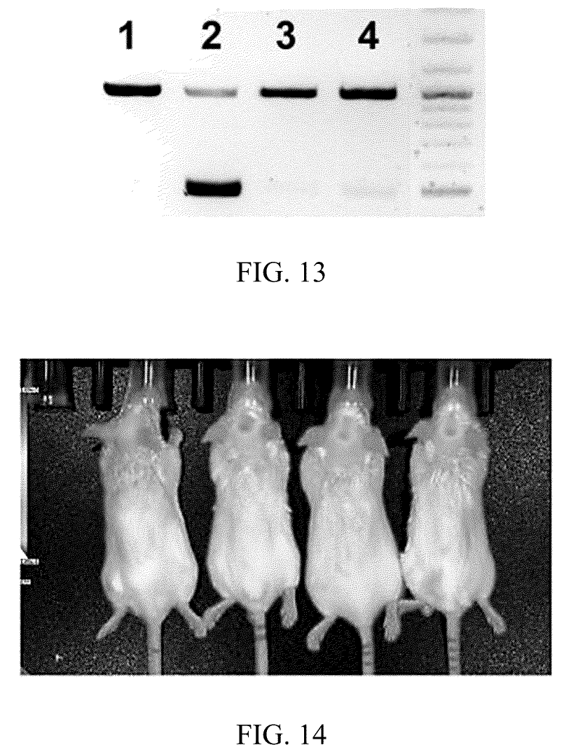

In a study, Cas9 guide ribonucleic acid (gRNA)-RNPs targeting mouse β2M were incubated with magnetite MNPs conjugated with (Arg)₁₂. After brief incubation, MNP was removed from the solution with a magnet. The number of RNPs left in the supernatant was examined by mixing the supernatant with the target DNA strand, incubating the mixture, and analyzing the resulting solution by gel electrophoresis. If RNPs were in the supernatant, it would be observed that the target strand is digested into short ones (FIG. 13, lane 2). Otherwise, RNPs were removed by MNP extraction, indicating good binding between RNPs and MNPs. Although MNPs with larger core sizes can improve magnetically driven endocytosis, they exhibit lower cargo loading capacity due to a decreased surface-area-to-volume ratio. To balance such effects, it is believed that MNPs with core sizes of 10 nm, 15 nm, and/or 20 nm may provide the desired cargo loading capacity while still facilitating good magnetically driven endocytosis (FIG. 13).

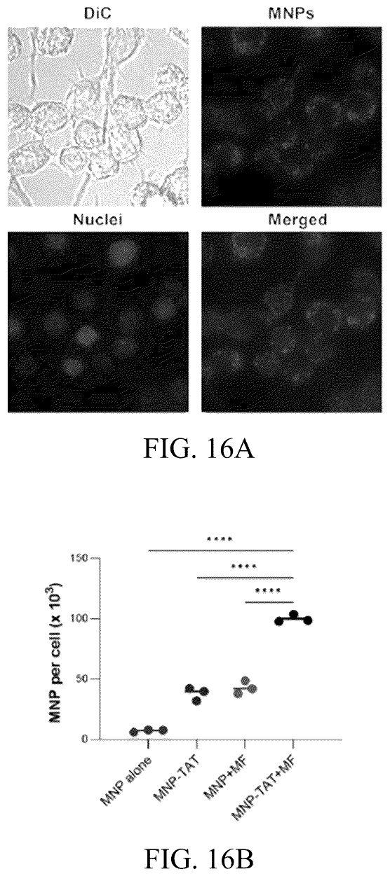

The combination effect of TAT and magnetic induction on endocytosis of MNPs was also examined. RAW264.7 cells were used as the model system because macrophages are known to produce exosomes that can traverse the BBB. RAW264.7 cells are incubated with a medium containing 50 μg/mL fluorophore labeled MNPs. As shown with fluorescence microscopy, the internalized MNPs were accumulated in small vesicles inside the cells (FIG. 16A). The amount of MNPs in the cells were measured using a colorimetric assay. Both TAT peptide conjugation and incubation over a magnetic plate could enhance the endocytosis of MNPs. When combined together, the uptake rate of MNPs increased from 29±4 MNP/minute to 416±14 MNP/minute for each cell (FIG. 16B).

These findings prove the feasibility of rapidly concentrating cargos in the endosomal compartment through MNP-enhanced endocytosis.

### Cell Homogenization and Vesicle Extraction

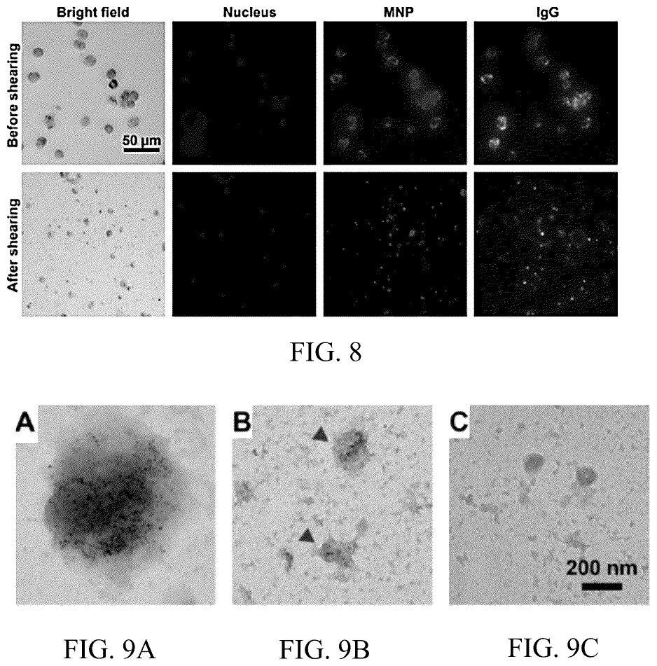

A cell homogenization and vesicle extraction protocol was established and designed to minimize genomic DNA contamination, which can activate DAMP receptor-mediated immune response. In a study, various homogenization techniques, including probe sonication, bath sonication, and mechanical shearing were compared. Shearing of RAW264.7 cells proved most effective, facilitating the release of intracellular vesicles while preserving nuclear integrity (FIG. 8). This is important for avoiding contamination by genomic DNA in the nuclei. Fragment of genomic DNA can be highly immunogenic and detrimental to downstream applications. The findings indicated that shearing RAW 264.7 cells through a 30G needle allowed for the release of intracellular vesicles while preserving the integrity of the cell nuclei. To ensure batch-to-batch consistency, a three-dimensionally-printed device that fixes the shearing syringe and collection tube in optimal positions was developed to standardize the shearing process (FIG. 5). Following homogenization, MNP-containing vesicles were isolated using a magnetic stand. A two-hour incubation was sufficient to recover most vesicles containing MNPs (FIG. 7). Notably, IgG molecules colocalized with MNPs within the extracted vesicles (small vesicles were observed moving in and out of the focal plane across image acquisitions).

### eEXO Production

RAW264.7 cells were incubated with MNP-TAT on a magnetic plate for four hours. Then the cells were detached from the plate and homogenized using the above-noted shearing protocol. The vesicles containing MNPs were collected from the cell homogenate with a magnetic plate. The vesicles were dispersed in an isotonic sucrose solution and sequentially passed through porous polycarbonate membranes of decreasing pore size (1000, 400, and 200 nm) installed in a Mini-Extruder. TEM confirmed the transition from large, heterogeneous vesicles (from several hundred nm to >1μm) containing abundant MNPs to uniform nanoscale eEXOs (˜100 nm) carrying small MNP clusters (FIGS. 9A and 9B). In this regard, it was found that after the vesicles undergo ten passes through the 200 nm pore-sized membrane, their size distribution closely approximates that of natural exosomes obtained via ultracentrifugation. As a control, the natural exosomes from the culture medium of matched RAW264.7 cells were collected via ultracentrifugation (FIG. 9C). Analysis using a nanoparticle tracking analysis (NTA) instrument showed that the eEXOs had a similar size distribution as that of natural exosomes, but the yield was increased significantly (FIGS. 9D and 9E). The eEXOs were also found to express CD63, a canonical exosome marker. Importantly, eEXO yield from RAW 264.7 cells increased by 46-fold (95% CI: 33-59) (FIG. 9F). These findings demonstrate that the integration of MNP-mediated cargo loading, endosomal vesicle enrichment, and membrane extrusion establishes a highly efficient workflow for scalable eEXO manufacturing. The resulting vesicles faithfully preserve the morphological and molecular hallmarks of natural exosomes while simultaneously overcoming the major barriers that have hindered clinical translation.

### Targeted Delivery via Surface Ligand Conjugation

Blood brain barrier (BBB) penetration can be significantly improved by decorating exosomes with specific peptide ligands. Among these, the rabies virus glycoprotein fragment RVG29 is well established to bind nicotinic acetylcholine receptors on brain microvascular endothelium, mediating receptor-dependent endocytosis. Once past the BBB, RVG29-exosomes can target neuronal cells by binding to acetylcholine receptors on their surface. Previous studies have demonstrated RVG29's capacity to facilitate retro-axonal and trans-synaptic spread, thereby improving the delivery of exosomes to neighboring neurons and promoting distribution within the central nervous system (CNS). Upon systemic administration of RVG29-exosomes carrying siRNAs, effective gene knockdown is observed in the striatum, midbrain, and cortex, with minimal impact on non-target tissues such as the kidney, liver, spleen, and heart.

A protocol linking RVG29 to a DSPE-PEG-DBCO copolymer via click chemistry has been developed. In brief, the RVG29 peptide can be synthesized with a terminal azidyl group and conjugated with DSPE-PEG via click chemistry. This lipid modified RVG29 can then be incorporated into the membrane of eEXOs through mixing and incubation in PBS. In a study, eEXOs conjugated with RVG29 and labeled with DiR were injected into the tail vein of three mice and compared against a control mouse provided PBS via tail vein injection. After 24 h, the mice were imaged with a small animal fluorescence imaging instrument. eEXOs modified with DSPE-PEG-RVG29 and labeled with the lipophilic dye DiR exhibited enhanced accumulation in the brain following IV injection (FIG. 14). These results validate both the conjugation chemistry and the feasibility of targeted eEXO delivery across the BBB.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Sander JD, Joung JK. CRISPR-Cas systems for editing, regulating and targeting genomes. Nat Biotechnol. 2014; 32(4):347-55. PubMed PMID: 24584096.
2. Canver MC, Smith EC, Sher F, Pinello L, Sanjana NE, Shalem O, et al. BCL11A enhancer dissection by Cas9-mediated in situ saturating mutagenesis. Nature. 2015; 527(7577):192-7. PubMed PMID: 26375006.
3. Wilkinson AC, Dever DP, Baik R, Camarena J, Hsu I, Charlesworth CT, et al. Cas 9-AAV6 gene correction of beta-globin in autologous HSCs improves sickle cell disease erythropoiesis in mice. Nat Commun. 2021; 12(1):686. PubMed PMID: 33514718.
4. Lattanzi A, Camarena J, Lahiri P, Segal H, Srifa W, Vakulskas CA, et al. Development of beta-globin gene correction in human hematopoietic stem cells as a potential durable treatment for sickle cell disease. Science translational medicine. 2021; 13(598). PubMed PMID: 34135108.
5. Cromer MK, Camarena J, Martin RM, Lesch BJ, Vakulskas CA, Bode NM, et al. Gene replacement of alpha-globin with beta-globin restores hemoglobin balance in beta-thalassemia-derived hematopoietic stem and progenitor cells. Nat Med. 2021; 27(4):677-87. PubMed PMID: 33737751.
6. De Ravin SS, Brault J, Meis RJ, Liu S, Li L, Pavel-Dinu M, et al. Enhanced homology-directed repair for highly efficient gene editing in hematopoietic stem/progenitor cells. Blood. 2021; 137(19):2598-608. PubMed PMID: 33623984.
7. Tong S, Moyo B, Lee CM, Leong K, Bao G. Engineered materials for in vivo delivery of genome-editing machinery. Nat Rev Mater. 2019; 4(11):726-37. PubMed PMID: 34094589.
8. Hsu PD, Scott DA, Weinstein JA, Ran FA, Konermann S, Agarwala V, et al. DNA targeting specificity of RNA-guided Cas9 nucleases. Nat Biotechnol. 2013; 31(9):827-32. PubMed PMID: 23873081.
9. Kalluri R, LeBleu VS. The biology, function, and biomedical applications of exosomes. Science. 2020; 367(6478):6977. PubMed PMID: 32029601.
10. O'Driscoll L. Expanding on exosomes and ectosomes in cancer. N Engl J Med. 2015; 372(24):2359-62. PubMed PMID: 26061842.
11. Raposo G, Stahl PD. Extracellular vesicles, genetic programmers. Nat Cell Biol. 2024; 26(1):22-3. PubMed PMID: 38228825.
12. Wu S, Yang T, Ma M, Fan L, Ren L, Liu G, et al. Extracellular vesicles meet mitochondria: Potential roles in regenerative medicine. Pharmacol Res. 2024; 206:107307. PubMed PMID: 39004243.
13. Alvarez-Erviti L, Seow Y, Yin H, Betts C, Lakhal S, Wood MJ. Delivery of siRNA to the mouse brain by systemic injection of targeted exosomes. Nat Biotechnol. 2011; 29(4):341-5. PubMed PMID: 21423189.
14. Di Ianni E, Obuchi W, Breyne K, Breakefield XO. Extracellular vesicles for the delivery of gene therapy. Nature Reviews Bioengineering. 2025; 3(5):360-73.
15. Yang Z, Shi J, Xie J, Wang Y, Sun J, Liu T, et al. Large-scale generation of functional mRNA-encapsulating exosomes via cellular nanoporation. Nat Biomed Eng. 2019. PubMed PMID: 31844155.
16. Lin Y, Wu J, Gu W, Huang Y, Tong Z, Huang L, et al. Exosome-Liposome Hybrid Nanoparticles Deliver CRISPR/Cas9 System in MSCs. Advanced Science. 2018; 5(4):1700611.
17. Cheng L, Hill AF. Therapeutically harnessing extracellular vesicles. Nat Rev Drug Discov. 2022; 21(5):379-99. PubMed PMID: 35236964.
18. Liu L, Li Y, Peng H, Liu R, Ji W, Shi Z, et al. Targeted exosome coating gene-chem nanocomplex as “nanoscavenger” for clearing alpha-synuclein and immune activation of Parkinson's disease. Sci Adv. 2020; 6(50). PubMed PMID: 33310840.
19. Wan T, Zhong J, Pan Q, Zhou T, Ping Y, Liu X. Exosome-mediated delivery of Cas 9 ribonucleoprotein complexes for tissue-specific gene therapy of liver diseases. Sci Adv. 2022; 8(37):eabp9435. PubMed PMID: 36103526.
20. Yao X, Lyu P, Yoo K, Yadav MK, Singh R, Atala A, et al. Engineered extracellular vesicles as versatile ribonucleoprotein delivery vehicles for efficient and safe CRISPR genome editing. J Extracell Vesicles. 2021; 10(5):e12076. PubMed PMID: 33747370.
21. Binenbaum Y, Fridman E, Yaari Z, Milman N, Schroeder A, Ben David G, et al. Transfer of miRNA in Macrophage-Derived Exosomes Induces Drug Resistance in Pancreatic Adenocarcinoma. Cancer Res. 2018; 78(18):5287-99. PubMed PMID: 30042153.
22. Kamerkar S, Leng C, Burenkova O, Jang SC, McCoy C, Zhang K, et al. Exosome-mediated genetic reprogramming of tumor-associated macrophages by exoASO-STAT6 leads to potent monotherapy antitumor activity. Sci Adv. 2022; 8(7):eabj7002. PubMed PMID: 35179953.
23. Cheng K, Kalluri R. Guidelines for clinical translation and commercialization of extracellular vesicles and exosomes based therapeutics. Extracellular Vesicle. 2023; 2:100029.
24. Welsh JA, Goberdhan DCI, O'Driscoll L, Buzas EI, Blenkiron C, Bussolati B, et al. Minimal information for studies of extracellular vesicles (MISEV2023): From basic to advanced approaches. J Extracell Vesicles. 2024; 13(2):e12404. PubMed PMID: 38326288.
25. Chen C, Sun M, Wang J, Su L, Lin J, Yan X. Active cargo loading into extracellular vesicles: Highlights the heterogeneous encapsulation behaviour. Journal of Extracellular Vesicles. 2021; 10(13).
26. Hettich BF, Bader JJ, Leroux JC. Encapsulation of Hydrophilic Compounds in Small Extracellular Vesicles: Loading Capacity and Impact on Vesicle Functions. Advanced healthcare materials. 2022; 11(5):e2100047. PubMed PMID: 33951319.
27. Liang Y, Duan L, Lu J, Xia J. Engineering exosomes for targeted drug delivery. Theranostics. 2021; 11(7):3183-95. PubMed PMID: 33537081.
28. Doudna JA. The promise and challenge of therapeutic genome editing. Nature. 2020; 578(7794):229-36. PubMed PMID: 32051598.
29. Cong L, Ran FA, Cox D, Lin SL, Barretto R, Habib N, et al. Multiplex Genome Engineering Using CRISPR/Cas Systems. Science. 2013; 339(6121):819-23. PubMed PMID: ISI: 000314874400049.
30. Maeder ML, Stefanidakis M, Wilson CJ, Baral R, Barrera LA, Bounoutas GS, et al. Development of a gene-editing approach to restore vision loss in Leber congenital amaurosis type 10. Nat Med. 2019; 25(2):229-33. PubMed PMID: 30664785.
31. Mitchell MJ, Billingsley MM, Haley RM, Wechsler ME, Peppas NA, Langer R. Engineering precision nanoparticles for drug delivery. Nat Rev Drug Discov. 2021; 20(2):101-24. PubMed PMID: 33277608.
32. Wang Y, Hu JK, Krol A, Li YP, Li CY, Yuan F. Systemic dissemination of viral vectors during intratumoral injection. Mol Cancer Ther. 2003; 2(11):1233-42. PubMed PMID: 14617797.
33. Mulcahy LA, Pink RC, Carter DR. Routes and mechanisms of extracellular vesicle uptake. J Extracell Vesicles. 2014; 3(1):24641. PubMed PMID: 25143819.
34. Wiklander OP, Nordin JZ, O'Loughlin A, Gustafsson Y, Corso G, Mager I, et al. Extracellular vesicle in vivo biodistribution is determined by cell source, route of administration and targeting. J Extracell Vesicles. 2015; 4(1):26316. PubMed PMID: 25899407.
35. Hoshino A, Costa-Silva B, Shen TL, Rodrigues G, Hashimoto A, Tesic Mark M, et al. Tumour exosome integrins determine organotropic metastasis. Nature. 2015; 527(7578):329- PubMed PMID: 26524530.
36. Teng F, Fussenegger M. Shedding Light on Extracellular Vesicle Biogenesis and Bioengineering. Adv Sci (Weinh). 2020; 8(1):2003505. PubMed PMID: 33437589.
37. Kojima R, Bojar D, Rizzi G, Hamri GC, El-Baba MD, Saxena P, et al. Designer exosomes produced by implanted cells intracerebrally deliver therapeutic cargo for Parkinson's disease treatment. Nat Commun. 2018; 9(1):1305. PubMed PMID: 29610454.
38. Kim HY, Kim TJ, Kang L, Kim Y-J, Kang MK, Kim J, et al. Mesenchymal stem cell-derived magnetic extracellular nanovesicles for targeting and treatment of ischemic stroke. Biomaterials. 2020; 243:119942.
39. Dar GH, Mendes CC, Kuan WL, Speciale AA, Conceicao M, Gorgens A, et al. GAPDH controls extracellular vesicle biogenesis and enhances the therapeutic potential of EV mediated siRNA delivery to the brain. Nat Commun. 2021; 12(1):6666. PubMed PMID: 34795295.
40. Kamerkar S, LeBleu VS, Sugimoto H, Yang S, Ruivo CF, Melo SA, et al. Exosomes facilitate therapeutic targeting of oncogenic KRAS in pancreatic cancer. Nature. 2017; 546(7659):498-503. PubMed PMID: 28607485.
41. Mendt M, Kamerkar S, Sugimoto H, McAndrews KM, Wu CC, Gagea M, et al. Generation and testing of clinical-grade exosomes for pancreatic cancer. JCI Insight. 2018; 3(8). PubMed PMID: 29669940.
42. Usman WM, Pham TC, Kwok YY, Vu LT, Ma V, Peng B, et al. Efficient RNA drug delivery using red blood cell extracellular vesicles. Nat Commun. 2018; 9(1):2359. PubMed PMID: 29907766.
43. Pottash A E, Levy D, Jeyaram A, Kuo L, Kronstadt SM, Chao W, et al. Combinatorial microRNA Loading into Extracellular Vesicles for Increased Anti-Inflammatory Efficacy. Noncoding RNA. 2022; 8(5) 71. PubMed PMID: 36287123.
44. Shtam TA, Kovalev RA, Varfolomeeva EY, Makarov EM, Kil YV, Filatov MV. Exosomes are natural carriers of exogenous siRNA to human cells in vitro. Cell Communication and Signaling. 2013; 11(1):88.
45. Jeyaram A, Lamichhane TN, Wang S, Zou L, Dahal E, Kronstadt SM, et al. Enhanced Loading of Functional miRNA Cargo via pH Gradient Modification of Extracellular Vesicles. Mol Ther. 2020; 28(3):975-85. PubMed PMID: 31911034.
46. Liang X, Gupta D, Xie J, Van Wonterghem E, Van Hoecke L, Hean J, et al. Engineering of extracellular vesicles for efficient intracellular delivery of multimodal therapeutics including genome editors. Nat Commun. 2025; 16(1):4028. PubMed PMID: 40301355.
47. Neupane KR, Ramon GS, Harvey B, Chun B, Aryal SP, Masud AA, et al. Programming Cell-Derived Vesicles with Enhanced Immunomodulatory Properties. Advanced healthcare materials. 2023; 12(27):e2301163. PubMed PMID: 37377147.
48. Lee JR, Park BW, Kim J, Choo YW, Kim HY, Yoon JK, et al. Nanovesicles derived from iron oxide nanoparticles-incorporated mesenchymal stem cells for cardiac repair. Sci Adv. 2020; 6(18):eaaz0952. PubMed PMID: 32494669.
49. Yang X, Yi Z, Liang Y, Tong S. Magnetic Iron Oxide Nanoparticles Enhance Exosome Production by Upregulating Exosome Transport and Secretion Pathways. ACS Appl Mater Interfaces. 2024; 16(49):67235-45. PubMed PMID: 39582356.
50. Liang Q, Bie N, Yong T, Tang K, Shi X, Wei Z, et al. The softness of tumour-cell-derived microparticles regulates their drug-delivery efficiency. Nat Biomed Eng. 2019; 3(9):729-40. PubMed PMID: 31110292.
51. Cardoso MH, Hall MJ, Burgoyne T, Fale P, Storm T, Escrevente C, et al. Impaired Lysosome Reformation in Chloroquine-Treated Retinal Pigment Epithelial Cells. Investigative ophthalmology & visual science. 2023; 64(11):10. PubMed PMID: 37548963.
52. Tong S, Hou S, Ren B, Zheng Z, Bao G. Self-assembly of phospholipid-PEG coating on nanoparticles through dual solvent exchange. Nano Lett. 2011; 11(9):3720-6. PubMed PMID: 21793503.
53. Tong S, Quinto CA, Zhang L, Mohindra P, Bao G. Size-Dependent Heating of Magnetic Iron Oxide Nanoparticles. ACS Nano. 2017; 11(7):6808-16. PubMed PMID: 28625045.
54. Sun S, Zeng H, Robinson DB, Raoux S, Rice PM, Wang SX, et al. Monodisperse MFe2O4 (M=Fe, Co, Mn) nanoparticles. J Am Chem Soc. 2004; 126(1):273-9. PubMed PMID: 14709092.
55. Yang H, Ogawa T, Hasegawa D, Takahashi M. Synthesis and magnetic properties of monodisperse magnetite nanocubes. J Appl Phys. 2008; 103(7):07D526. PubMed PMID: ISI: 000255043200454.
56. Zhang LL, Tong S, Zhang QB, Bao G. Lipid-Encapsulated Fe3O4 Nanoparticles for Multimodal Magnetic Resonance/Fluorescence Imaging. Acs Applied Nano Materials. 2020; 3(7):6785-97. PubMed PMID: WOS: 000555518200063.
57. Comazzetto S, Shen B, Morrison SJ. Niches that regulate stem cells and hematopoiesis in adult bone marrow. Developmental Cell. 2021; 56(13):1848-60.
58. Acar M, Kocherlakota KS, Murphy MM, Peyer JG, Oguro H, Inra CN, et al. Deep imaging of bone marrow shows non-dividing stem cells are mainly perisinusoidal. Nature. 2015; 526(7571):126-30.
59. Luck RP, Haines C, Mull CC. Intraosseous Access. The Journal of Emergency Medicine. 2010; 39(4):468-75.
60. Worsham DN, Schuesler T, Von Kalle C, Pan D. In Vivo Gene Transfer into Adult Stem Cells in Unconditioned Mice by in Situ Delivery of a Lentiviral Vector. Molecular Therapy. 2006; 14(4):514-24.
61. Shi M, Adachi Y, Shigematsu A, Koike-Kiriyama N, Feng W, Yanai S, et al. Intra-bone marrow injection of donor bone marrow cells suspended in collagen gel retains injected cells in bone marrow, resulting in rapid hemopoietic recovery in mice. Stem Cells. 2008; 26(9):2211-6. PubMed PMID: 18599811.
62. Christodoulou C, Spencer JA, Yeh S-CA, Turcotte R, Kokkaliaris KD, Panero R, et al. Live-animal imaging of native haematopoietic stem and progenitor cells. Nature. 2020; 578(7794):278-83.
63. Kokkaliaris KD. Dissecting the spatial bone marrow microenvironment of hematopoietic stem cells. Current opinion in oncology. 2020; 32(2).
64. Kokkaliaris KD, Kunz L, Cabezas-Wallscheid N, Christodoulou C, Renders S, Camargo F, et al. Adult blood stem cell localization reflects the abundance of reported bone marrow niche cell types and their combinations. Blood. 2020; 136(20):2296-307.
65. Upadhaya S, Krichevsky O, Akhmetzyanova I, Sawai CM, Fooksman DR, Reizis B. Intravital Imaging Reveals Motility of Adult Hematopoietic Stem Cells in the Bone Marrow Niche. Cell stem cell. 2020; 27(2):336-45.e4.
66. Heil J, Olsavszky V, Busch K, Klapproth K, de la Torre C, Sticht C, et al. Bone marrow sinusoidal endothelium controls terminal erythroid differentiation and reticulocyte maturation. Nat Commun. 2021; 12(1):6963. PubMed PMID: 34845225.
67. Landazuri N, Tong S, Suo J, Joseph G, Weiss D, Sutcliffe DJ, et al. Magnetic targeting of human mesenchymal stem cells with internalized superparamagnetic iron oxide nanoparticles. Small. 2013; 9(23):4017-26. PubMed PMID: 23766267.
68. Zhang L, Hajebrahimi S, Tong S, Gao X, Cheng H, Zhang Q, et al. Force-Mediated Endocytosis of Iron Oxide Nanoparticles for Magnetic Targeting of Stem Cells. ACS Appl Mater Interfaces. 2023; 15(44):50574-85. PubMed PMID: 37145890.
69. Ben Haddada M, Hu D, Salmain M, Zhang L, Peng C, Wang Y, et al. Gold nanoparticle-based localized surface plasmon immunosensor for staphylococcal enterotoxin A (SEA) detection. Analytical and Bioanalytical Chemistry. 2017; 409(26):6227-34.
70. Wolfram J, Nizzero S, Liu H, Li F, Zhang G, Li Z, et al. A chloroquine-induced macrophage-preconditioning strategy for improved nanodelivery. Scientific reports. 2017; 7(1):13738. PubMed PMID: 29062065.
71. Kumar P, Ban HS, Kim SS, Wu H, Pearson T, Greiner DL, et al. T cell-specific siRNA delivery suppresses HIV-1 infection in humanized mice. Cell. 2008; 134(4):577-86. PubMed PMID: 18691745.
72. Fredericksen BL, Wei BL, Yao J, Luo T, Garcia JV. Inhibition of endosomal/lysosomal degradation increases the infectivity of human immunodeficiency virus. J Virol. 2002; 76(22):11440-6. PubMed PMID: 12388705.
73. Bayer N, Schober D, Prchla E, Murphy RF, Blaas D, Fuchs R. Effect of bafilomycin A1 and nocodazole on endocytic transport in HeLa cells: implications for viral uncoating and infection. J Virol. 1998; 72(12):9645-55. PubMed PMID: 9811698.
74. Runsala M, Kuokkanen E, Uski E, Sustar V, Balci MO, Rajala J, et al. The Small GTPase Rab7 Regulates Antigen Processing in B Cells in a Possible Interplay with Autophagy Machinery. Cells. 2023; 12(21). PubMed PMID: 37947644.
75. Théry C, Witwer KW, Aikawa E, Alcaraz MJ, Anderson JD, Andriantsitohaina R, et al. Minimal information for studies of extracellular vesicles 2018 (MISEV2018): a position statement of the International Society for Extracellular Vesicles and update of the MISEV 2014 guidelines. J Extracell Vesicles. 2018; 7(1):1535750. PubMed PMID: 30637094.
76. Bonsergent E, Grisard E, Buchrieser J, Schwartz O, Thery C, Lavieu G. Quantitative characterization of extracellular vesicle uptake and content delivery within mammalian cells. Nat Commun. 2021; 12(1):1864. PubMed PMID: 33767144.
77. Dowdy SF, Setten RL, Cui XS, Jadhav SG. Delivery of RNA Therapeutics: The Great Endosomal Escape! Nucleic acid therapeutics. 2022; 32(5):361-8. PubMed PMID: 35612432.
78. Zhao X, Zhang C, Cui X, Liang Y. Interactions of Hematopoietic Stem Cells with Bone Marrow Niche. Methods Mol Biol. 2021; 2346:21-34. PubMed PMID: 32504296.
79. Cui X, Hou L, Yan B, Liu J, Zhang C, Sui P, et al. Sexual dimorphism in the mouse bone marrow niche regulates hematopoietic engraftment via sex-specific Kdm5c/Cxcl12 signaling. The Journal of clinical investigation. 2025; 135(5). PubMed PMID: 39836478.
80. Kumar P, Wu H, McBride JL, Jung KE, Kim MH, Davidson BL, et al. Transvascular delivery of small interfering RNA to the central nervous system. Nature. 2007; 448(7149):39-43. PubMed PMID: 17572664.
81. Fitzhugh CD, Cordes S, Taylor T, Coles W, Roskom K, Link M, et al. At least 20% donor myeloid chimerism is necessary to reverse the sickle phenotype after allogeneic HSCT. Blood. 2017; 130(17):1946-8.
82. Pan D. In situ (in vivo) gene transfer into murine bone marrow stem cells. Methods Mol Biol. 2009; 506:159-69. PubMed PMID: 19110626.
83. Newby GA, Yen JS, Woodard KJ, Mayuranathan T, Lazzarotto CR, Li Y, et al. Base editing of haematopoietic stem cells rescues sickle cell disease in mice. Nature. 2021; 595(7866):295-302. PubMed PMID: 34079130.
84. Zhu H, Zhang L, Tong S, Lee CM, Deshmukh H, Bao G. Spatial control of in vivo CRISPR-Cas 9 genome editing via nanomagnets. Nat Biomed Eng. 2019; 3(2):126-36. PubMed PMID: 30944431.
85. Stuart T, Butler A, Hoffman P, Hafemeister C, Papalexi E, Mauck WM, 3rd, et al. Comprehensive Integration of Single-Cell Data. Cell. 2019; 177(7):1888-902 e21. PubMed PMID: 31178118.
86. Finak G, McDavid A, Yajima M, Deng J, Gersuk V, Shalek AK, et al. MAST: a flexible statistical framework for assessing transcriptional changes and characterizing heterogeneity in single-cell RNA sequencing data. Genome Biol. 2015; 16:278. PubMed PMID: 26653891.
87. Subramanian A, Tamayo P, Mootha VK, Mukherjee S, Ebert BL, Gillette MA, et al. Gene set enrichment analysis: a knowledge-based approach for interpreting genome-wide expression profiles. Proc Natl Acad Sci U S A. 2005; 102(43):15545-50. PubMed PMID: 16199517.
88. Heidarzadeh M, Gursoy-Ozdemir Y, Kaya M, Eslami Abriz A, Zarebkohan A, Rahbarghazi R, Sokullu E. Exosomal delivery of therapeutic modulators through the blood-brain barrier; promise and pitfalls. Cell Biosci. 2021; 11(1):142. Epub 20210722. doi: 10.1186/s13578-021-00650-0. PubMed PMID: 34294165; PMCID: PMC8296716.
89. Shtam TA, Kovalev RA, Varfolomeeva EY, Makarov EM, Kil YV, Filatov MV. Exosomes are natural carriers of exogenous siRNA to human cells in vitro. Cell Communication and Signaling. 2013; 11(1):88. doi: 10.1186/1478-811X-11-88.
90. Tong S, Zhu H, Bao G. Magnetic iron oxide nanoparticles for disease detection and therapy. Materials Today. 2019; 31:86-99. doi: 10.1016/j.mattod.2019.06.003.
91. Boutin S, Monteilhet V, Veron P, Leborgne C, Benveniste O, Montus MF, Masurier C. Prevalence of serum IgG and neutralizing factors against adeno-associated virus (AAV) types 1, 2, 5, 6, 8, and 9 in the healthy population: implications for gene therapy using AAV vectors. Hum Gene Ther. 2010; 21(6): 704-12. doi: 10.1089/hum.2009.182. PubMed PMID: 20095819.
92. Sun W, Ji W, Hall JM, Hu Q, Wang C, Beisel CL, Gu Z. Self-assembled DNA nanoclews for the efficient delivery of CRISPR-Cas9 for genome editing. Angew Chem Int Ed Engl. 2015; 54(41):12029-33. doi: 10.1002/anie.201506030. PubMed PMID: 26310292; PMCID: PMC4677991.
93. Tong S, Ren B, Zheng Z, Shen H, Bao G. Tiny grains give huge gains: nanocrystal-based signal amplification for biomolecule detection. ACS Nano. 2013; 7(6):5142-50. Epub 2013 May 11. doi: 10.1021/nn400733t. PubMed PMID: 23659350; PMCID: 3758924.
94. Wu T, Liu Y, Cao Y, Liu Z. Engineering Macrophage Exosome Disguised Biodegradable Nanoplatform for Enhanced Sonodynamic Therapy of Glioblastoma. Adv Mater. 2022; 34(15): e2110364. Epub 20220227. doi: 10.1002/adma.202110364. PubMed PMID: 35133042.
95.Yuan D, Zhao Y, Banks WA, Bullock KM, Haney M, Batrakova E, Kabanov AV. Macrophage exosomes as natural nanocarriers for protein delivery to inflamed brain. Biomaterials. 2017; 142:1-12. doi: 10.1016/j.biomaterials.2017 Jul. 11.
96. Huotari J, Helenius A. Endosome maturation. The EMBO Journal. 2011; 30(17):3481-500. doi: 10.1038/emboj.2011.286.
97. Cullen PJ, Steinberg F. To degrade or not to degrade: mechanisms and significance of endocytic recycling. Nature reviews Molecular cell biology. 2018; 19(11):679-96. doi: 10.1038/s41580-018-0053-7. Pubmed Pmid: 30194414.
98. Cui GH, Guo HD, Li H, Zhai Y, Gong ZB, Wu J, et al. RVG-modified exosomes derived from mesenchymal stem cells rescue memory deficits by regulating inflammatory responses in a mouse model of Alzheimer's disease. Immun Ageing. 2019; 16(1):10. Epub 20190513. doi: 10.1186/s12979-019-0150-2. PubMed PMID: 31114624; PMCID: PMC6515654.
99. Théry C, Witwer KW, Aikawa E, Alcaraz MJ, Anderson JD, Andriantsitohaina R, et al. Minimal information for studies of extracellular vesicles 2018 (MISEV2018): a position statement of the International Society for Extracellular Vesicles and update of the MISEV 2014 guidelines. J Extracell Vesicles. 2018; 7(1):1535750. Epub 20181123. doi: 10.1080/20013078.2018.1535750. PubMed PMID: 30637094; PMCID: PMC6322352.
100. Ciftci E, Bozbeyoglu N, Gursel I, Korkusuz F, Bakan Misirlioglu F, Korkusuz P. Comparative analysis of magnetically activated cell sorting and ultracentrifugation methods for exosome isolation. PLoS One. 2023; 18(2): e0282238. Epub 20230228. doi: 10.1371/journal. pone.0282238. PubMed PMID: 36854030; PMCID: PMC9974127.
101. Wang YX. Superparamagnetic iron oxide based MRI contrast agents: Current status of clinical application. Quantitative imaging in medicine and surgery. 2011; 1(1):35-40. Epub 2012 Dec. 21. doi: 10.3978/j.issn.2223-4292.2011.08.03. PubMed PMID: 23256052; PMCID: PMC3496483.
102. Qiu Y, Tong S, Zhang L, Sakurai Y, Myers DR, Hong L, et al. Magnetic forces enable controlled drug delivery by disrupting endothelial cell-cell junctions. Nat Commun. 2017; 8:15594. Epub 2017 Jun. 9. doi: 10.1038/ncomms 15594. PubMed PMID: 28593939; PMCID: PMC5472756.
103. U.S. Patent Application Publication No. 2025/0145975, “Methods for In Vivo Delivery and Detection of Clustered Regularly Interspersed Short Palindromic Repeat Systems Utilizing Baculovirus Vector-Magnetic Nanoparticle Complexes, published May 8, 2025.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for exosome production, comprising:
complexing a therapeutic payload with one or more magnetic nanoparticles (MNPs) to form a MNP-therapeutic payload complex;
loading the MNP-therapeutic payload complex into an endosome of a producer cell;
extracting the endosome containing the MNP-therapeutic payload complex from the producer cell; and
extruding the extracted endosome containing the MNP-therapeutic payload complex to produce an engineered exosome.

2. The method of claim 1, wherein the therapeutic payload is a clustered regularly interspersed short palindromic repeat (CRISPR) ribonucleoprotein (RNP), and wherein the CRISPR RNP and the one or more MNPs form a MNP-RNP complex.

3. The method of claim 2, wherein the CRISPR RNP is selected from the group consisting of a CRISPR associated protein 9 (Cas9)-guide ribonucleic acid (gRNA) RNP, a base editor-gRNA RNP, and a prime editor-prime editing gene (PEG) RNA RNP.

4. The method of claim 1, wherein the one or more MNPs are conjugated to one or more cell-penetrating peptides.

5. The method of claim 4, wherein the one or more cell penetrating peptides includes a poly-argenine peptide, a transactivating transcriptional activator (TAT) peptide, or a combination thereof.

6. The method of claim 1, wherein each of the one or more MNPs have a core comprising magnetite.

7. The method of claim 1, wherein the one or MNPs has a core that is coated with 2-Distearoyl-sn-glycero-3-phosphoethanolamine-Poly(ethylene glycol) (DSPE-PEG).

8. The method of claim 1, wherein loading the MNP-therapeutic payload complex into the endosome includes applying a magnetic force to the MNP-therapeutic payload complex using one or more magnets.

9. The method of claim 1, wherein extracting the endosome containing the MNP-therapeutic payload complex from the producer cell includes applying a magnetic force to the MNP-therapeutic payload complex using one or more magnets.

10. The method of claim 9, wherein extracting the endosome containing the MNP-therapeutic payload complex from the producer cell includes homogenizing the producer cell prior to applying the magnetic force to the MNP-therapeutic payload complex.

11. The method of claim 10, wherein the producer cell is homogenized via mechanical shearing.

12. The method of claim 1, wherein extruding the extracted endosome includes passing the extracted endosome through one or more porous membranes.

13. The method of claim 12, wherein extruding the extracted endosome includes sequentially passing the extracted exosome through multiple porous membranes of decreasing size.

14. The method of claim 12, wherein each membrane of the one or more porous membranes has a pore size ranging from 200 nm to 1000 nm.

15. The method of claim 1, and further comprising conjugating at least one of a glycoprotein fragment and a fugogenic peptide to the engineered exosome.

16. The method of claim 1, wherein the therapeutic payload is an IgG antibody.

17. A method for exosome production, comprising:
complexing a clustered regularly interspersed short palindromic repeat (CRISPR) ribonucleoprotein (RNP) with one or more magnetic nanoparticles (MNPs) to form a MNP-RNP complex;
loading the MNP-RNP complex into an endosome of a producer cell at least in part by applying a first magnetic force to the MNP-RNP complex;
homogenizing the producer cell;
extracting the endosome containing the MNP-RNP complex from the homogenized producer cell at least in part by applying a second magnetic force to the MNP-RNP complex;
extruding the extracted endosome containing the MNP-RNP complex through one or more porous membranes to produce an engineered exosome.

18. The method of claim 1, wherein the CRISPR RNP is a CRISPR associated protein 9 (Cas9)-guide ribonucleic acid (gRNA) RNP.

19. An engineered exosome, comprising:
a therapeutic payload;
one or more MNPs complexed to the therapeutic payload, such that the MNPs and the therapeutic payload form a MNP-therapeutic payload complex; and
a vesicle containing the MNP-therapeutic payload complex.

20. The engineered exosome of claim 19, wherein the therapeutic payload is a IgG antibody, a clustered regularly interspersed short palindromic repeat (CRISPR) associated protein 9 (Cas9)-guide ribonucleic acid (gRNA) ribonucleoprotein (RNP), base editor-gRNA RNP, or a prime editor-prime editing gene (PEG) RNA RNP.
